(19) United States
(12) Patent Application Publication
(10) Pub. No.: US 2008/0274944 A1
(43) Pub. Date: Nov. 6, 2008

(54) Sorbol Esters as Perfuming Ingredients

(76) Inventors: Christian Margot, Gilly (CH); Jean-Marc Gaudin, Annemasse (FR); Hubert Mimoun, Challex (FR)

Correspondence Address: WINSTON & STRAWN LLP;PATENT DEPARTMENT, 1700 K STREET, N.W., WASHINGTON, DC, 20006, US

(21) Appl. No.: 12/088,054

(22) PCT Filed: Nov. 3, 2006
(86) PCT No.: PCT/IB2006/054103

§ 371 Date: Mar. 25, 2008

(30) Foreign Application Priority Data
Nov. 17, 2005 (IB) PCTIB2005/053799

(51) Int. Cl. A61K 8/37; A61Q 13/00

(52) U.S. Cl. 512/26; 560/129

(57) ABSTRACT

The present invention discloses perfuming ingredients, which are unsaturated esters of sorbol, as well as the use of such compounds in the perfumery industry and in compositions or articles containing such compounds.

### TECHNICAL FIELD

The present invention relates to the field of perfumery. More particularly, it concerns perfuming ingredients which are unsaturated esters of sorbol.

The present invention concerns the use of said compounds in the perfumery industry as well as the compositions or articles containing said compounds.

### PRIOR ART

Amongst the compounds of formula (I) only one is known in the prior art, namely 2,4-hexadienyl sorbate (see U.S. Pat. No. 3,283,032). This compound is described as a chemical useful in the context of thermosetting resins made from diene compounds. In the prior art document there is no mention of the organoleptic properties of the invention's compound.

To the best of our knowledge, only one sorbol ester is known as perfuming ingredients from the prior art. This compound is the sorbol isobutyrate described by S. Arctander (No 1578, in Perfume and Flavor Chemicals, 1969, Montclair, N.J., USA) as having an oily-sweet, apple and galbanum-like odor. The organoleptic properties of said prior art compound are quite different from the ones of the present compounds.

### DESCRIPTION OF THE INVENTION

We have now surprisingly discovered that a compound of formula

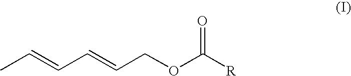

wherein R represents a linear C₅ alkenyl or alkadienyl group, a 6-hydroxy-phenyl group or a (C₆H₄)CH₂CH₂ group can be used as perfuming ingredient, for instance to impart odor notes of the floral or pear type.

According to a particular embodiment of the invention, a compound of formula (I) wherein R represents a C₅ alkenyl group or a 6-hydroxy-phenyl group is particularly appreciated.

Amongst the compounds of formula (I), one may cite in particular, and as non-limiting example, (2E,4E)-2,4-hexadienyl salicylate. This compound has a floral odor with a powdery, mimosa and slightly ethereal character. The odor of this compound possesses also an interesting vanilla, maple syrup and frangipani bottom note that may allow the introduction of original olfactif effects to articles or compositions in which the invention's compound is to be added. This compound, according to an embodiment of the invention, is particularly appreciated.

Another example of invention's compounds is (E,E)-2,4-hexadienyl (Z)-3-hexenoate which possesses typically pear odor with a sparkling/tingling green note. Although having a fruity type odor, the present compound differs significantly in character from the odor of the sorbol ester of the prior art (i.e the sorbol isobutyrate). Indeed, the present (Z)-3-hexenoate differs from the isobutyrate by not having the apple, resin and mastic odor, nor the fatty green note, of the prior art compound.

Yet a further example of the invention is (2E,4E)-2,4-hexadienyl 3-phenyl-propanoate, which is characterized by a floral-hyacinth odor with a honey-cinnamic like bottom note.

None of the invention's compounds is usable to impart apple-galbanum odors.

The compounds of formula (I) wherein R represents a linear C₅ alkenyl group, a 6-hydroxy-phenyl group or a (C₆H₄)CH₂CH₂ group, are also new compounds and therefore are also an object of the present invention.

As mentioned above, the invention concerns the use of a compound of formula (I) as perfuming ingredients. In other words it concerns a method to confer, enhance, improve or modify the odor properties of a perfuming composition or of a perfumed article, which method comprises adding to said composition or article an effective amount of at least a compound of formula (I). In particular the compounds (I) can be used to impart floral or pear type odor notes.

By “use of a compound of formula (I)” it has to be understood here also the use of any composition containing at least one compound (I) and which can be advantageously employed in perfumery industry as active ingredients.

Said composition, which in fact can be advantageously employed as perfuming ingredient, is also an object of the present invention.

Therefore, another object of the present invention is a perfuming composition comprising:
i) as perfuming ingredient, at least one invention's compound as defined above;
ii) at least one ingredient selected from the group consisting of a perfumery carrier and a perfumery base; and
iii) optionally at least one perfumery adjuvant.

By “perfumery carrier” we mean here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting example solvents such as dipropyleneglycol, diethyl phthalate, isopropyl myristate, benzyl benzoate, 2-(2-ethoxyethoxy)-1-ethanol or ethyl citrate, which are the most commonly used.

As solid carrier one may cite, as non-limiting examples, absorbing gums or polymers, or yet encapsulating materials. Examples of such materials, for example, may comprise wall-forming and plasticizing materials, such as mono-, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloids: Stabilisatoren, Dickungs-und Gehermittel in Lebensmittel, Band 2 der Schriftenreihe Lebensmittelchemie, Lebensmittelqualität, Behr's VerlagGmbH & Co., Hamburg, 1996. The encapsulation is a well known process to a person skilled in the art, and may be performed, for instance, using techniques such as spray-drying, agglomeration or yet extrusion; or consists of a coating encapsulation, including coacervation and complex coacervation techniques.

By “perfumery base” we mean here a composition comprising at least one perfuming co-ingredient.

Said perfuming co-ingredient is not of the formula (I). Moreover, by “perfuming co-ingredient” it is meant here a compound, which is used in perfuming preparation or composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of its general knowledge and according to intended use or application and the desired organoleptic effect. In general terms, these perfuming co-ingredients belong to chemical classes as varied as alcohols, aldehydes, ketones, esters, ethers, acetates, nitrites, terpene hydrocarbons, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

For the compositions which comprise both a perfumery carrier and a perfumery base, other suitable perfumery carrier, than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company).

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

An invention's composition consisting of at least one compound of formula (I) and at least one perfumery carrier represents a particular embodiment of the invention as well as a perfuming composition comprising at least one compound of formula (I), at least one perfumery carrier, at least one perfumery base, and optionally at least one perfumery adjuvant.

It is useful to mention here that the possibility to have, in the compositions mentioned above, more than one compound of formula (I) is important as it enables the perfumer to prepare accords, perfumes, possessing the odor tonality of various compounds of the invention, creating thus new tools for their work.

Its is also understood here that, unless otherwise indicated or described, any mixture resulting directly from a chemical synthesis, e.g. without an adequate purification, in which the compound of the invention would be involved as a starting, intermediate or end-product could not be considered as a perfuming composition according to the invention.

Furthermore, the invention's compound can also be advantageously used in all the fields of modern perfumery to positively impart or modify the odor of a consumer product into which said compound (I) is added. Consequently, a perfumed article comprising:

i) as perfuming ingredient, at least one compound of formula (I), as defined above; and
ii) a consumer product base,
is also an object of the present invention.

For the sake of clarity, it has to be mentioned that, by “consumer product base” we mean here a consumer product, which is compatible with perfuming ingredients. In other words, a perfumed article according to the invention comprises the functional formulation, as well as optionally additional benefit agents, corresponding to a consumer product, e.g. a detergent or an air freshener, and an olfactive effective amount of at least one invention's compound.

The nature and type of the constituents of the consumer product do not warrant a more detailed description here, which in any case would not be exhaustive, the skilled person being able to select them on the basis of its general knowledge and according to the nature and the desired effect of said product.

Examples of suitable consumer products include solid or liquid detergents and fabric softeners as well as all the other articles common in perfumery, namely perfumes, colognes or after-shave lotions, perfumed soaps, shower or bath salts, mousses, oils or gels, hygiene products or hair care products such as shampoos, body-care products, deodorants or antiperspirants, air fresheners and also cosmetic preparations. As detergents there are intended applications such as detergent compositions or cleaning products for washing up or for cleaning various surfaces, e.g. intended for textile, dish or hard-surface treatment, whether they are intended for domestic or industrial use. Other perfumed articles are fabric refreshers, ironing waters, papers, wipes or bleaches.

Some of the above-mentioned consumer product bases may represent an aggressive medium for the invention's compound, so that it may be necessary to protect the latter from premature decomposition, for example by encapsulation.

The proportions in which the compounds according to the invention can be incorporated into the various aforementioned articles or compositions vary within a wide range of values. These values are dependent on the nature of the article to be perfumed and on the desired organoleptic effect as well as the nature of the co-ingredients in a given base when the compounds according to the invention are mixed with perfuming co-ingredients, solvents or additives commonly used in the art.

For example, in the case of perfuming compositions, typical concentrations are in the order of 0.001% to 10% by weight, or even more, of the compounds of the invention based on the weight of the composition into which they are incorporated. Concentrations lower than these, such as in the order of 0.01% to 5% by weight, can be used when these compounds are incorporated into perfumed articles, percentage being relative to the weight of the article.

### EXAMPLES

The invention will now be described in further detail by way of the following examples, wherein the abbreviations have the usual meaning in the art, the temperatures are indicated in degrees centigrade (° C.); the NMR spectral data were recorded in CDCl₃ (if not stated otherwise) with a 360 or 400 MHz machine for ¹H and ¹³C, the chemical displacements δ are indicated in ppm with respect to TMS as standard, the coupling constants J are expressed in Hz.

### Example 1

### Preparation of Compounds of Formula (I)

### A) Synthesis of (E,E)-2,4-hexadienyl (Z)-3-hexenoate

Butyl (Z)-3-hexenoate (255 g, 1.5 mole), sorbol (177 g, 1.8 mole) and Ti(OⁱPr)₄ (11.25 g) were charged in a round bottom flask equipped with a distillation apparatus and were stirred at 130° C. for 2 hours, while distilling the butanol formed. Then the temperature was lowered to 90° C., and maintained during 6 hours (the butanol formed was distilled off at a pressure of 100 mbar). Finally the pressure was lowered to about 10 mbar and the residual sorbol and butyl (Z)-3-hexenoate was distilled of. The title compound was obtained by distillation of the residue (10 mbar, b.p. 110° C.) (yield 85%).

¹³C-NMR: 13.9; 18.1; 20.7; 32.9; 65.1; 120.2; 123.7; 130.5; 131.2; 134.9; 135.1; 171.8

### B) Synthesis of (E,E)-2,4-hexadienyl 3-phenylpropanoate

With a syringe, 11.2 g (66 mmol.) of hydrocinnamoyl chloride was slowly added to a magnetically stirred solution of 5 g (51 mmol.) of sorbol and 10.3 g (67 mmol.) of triethylamine in approximately 70 ml of dichloromethane. For this addition the exotherm is controlled by an ice bath, then the reaction was stirred overnight at room temperature.

The reaction was then hydrolyzed. The organic solution was washed three times with a 10% HCl solution and with water until neutral pH, dried over magnesium sulfate and concentrated. The crude compound was then distilled (b.p.=140-150° C. under 0.4 mbar), afforded 9 g of pure product (76% yield).

¹³C-NMR: 18.1 (q); 30.9 (t); 35.9 (t); 64.9 (t); 123.7 (d); 126.2 (d); 128.3 (d, 2C) 128.5 (d, 2C); 130.5 (d); 131.2 (d); 134.9 (d); 140.5 (s); 172.6 (s).

¹H-NMR: 1.75 (d, J=7, 3H); 2.62 (t, J=8, 2H); 2.95 (t, J=8, 2H); 4.57 (d, J=8, 2H) 5.58 (dt, J=14, J=7, 1H); 5.73 (dq, J=15 Hz, J=6, 1H); 6.04 (dd, J=15, J=10, 1H); 6.21 (dd, J=15, J=10, 1H); 7.15-7.20 (m, 3H); 7.25-7.30 (m, 2H).

### C) Synthesis of (2E,4E)-2,4-hexadienyl salicylate

In a Schlenk tube under argon, (E,E)-2,4-hexadien-1-ol (4.3 g, 44 mmol) is diluted with 50 ml anhydrous THF. At −20°, butyllithium 1.6M in hexane (27.5 ml, 44 mmol) is added dropwise over 10 min. The mixture is allowed to reach 20° within 20 minutes.

A solution of phenyl salicylate (9.4 g, 44 mmol) in 50 ml anhydrous THF is added over 10 min. After 15 hours, the reaction mixture is poured onto 500 ml ice water. Decantation, extraction of the aqueous phase with 3×50 ml diethyl ether, washing of the combined organic phases with brine, drying and solvent evaporation leaves a crude oil. The product is recrystallized from pentane at −20°. Yield 4.4 g (46%).

¹H-NMR: 10.78 (s, 1H); 7.85 (dd, J=2; 8, 1H); 7.44 (dt, J=2; 8, 1H); 6.97 (d, J=8.5, 1H); 6.86 (t, J=8, 1H); 6.34 (dd, J=11; 15, 1H); 6.08 (dd, J=10; 15, 1H); 5.75 (m, 2H); 4.84 (d, J=6.5, 2H); 1.77 (d, J=7, 3H).

¹³C-NMR: 170.0 (s); 161.7 (s); 135.7 (d); 135.6 (d); 131.9 (d); 130.3 (d); 130.0 (d); 122.9 (d); 119.1 (d); 117.5 (d); 112.5 (s); 65.8 (t); 18.2 (q).

### Example 2

### Preparation of a Perfuming Composition

A perfuming composition of the floral-herbaceous-spicy and balsamic type was prepared by admixing the following ingredients

| Ingredient | Parts by weight |
|---|---|
| 10%* Amyl acetate | 5 |
| Benzyl acetate | 5 |
| Carbinol BDM acetate | 5 |
| Linalyl acetate | 100 |
| Styrallyl acetate | 5 |
| 50%* Cinnamic alcohol | 15 |
| 10%* Anisic aldehyde | 5 |
| 10%* C 11 LENIQUE aldehyde | 10 |
| 10%* C 12 aldehyde | 5 |
| Hexylcinnamic aldehyde | 45 |
| 1%* Phenylacetic aldehyde | 45 |
| 1%* Cetalox ®¹⁾ Laevo | 15 |
| Lemon essential oil | 40 |
| Eugenol | 10 |
| 10%* Galbanum essential oil | 10 |
| Habanolide ®²⁾ | 100 |
| Iralia Total ®³⁾ | 60 |
| ISO E SUPER ®⁴⁾ | 45 |
| Isoeugenol | 10 |
| Jasmin essential oil | 15 |
| Linalool | 45 |
| Lorysia ®⁵⁾ | 10 |
| Lyral ®⁶⁾ | 50 |
| Mandarin essential oil | 15 |
| 1%* Crystal moss | 15 |
| Muscenone⁷⁾ delta | 5 |
| 10%* Cis-Jasmone | 10 |
| Hedione ®⁸⁾ | 70 |
| Phenethylol | 80 |
| Vertofix ®⁹⁾ coeur | 15 |
| Vetyver acetyl | 10 |
| Violet essential oil | 5 |
| Ylang | 20 |
| | 900 |

*in dipropyleneglycol
¹⁾dodecahydro-3a,6,6,9a-tetramethyl-naphtho[2,1-b]furan; origin: Firmenich SA, Switzerland
²⁾pentadecenolide; origin: Firmenich SA, Switzerland
³⁾mixture of isomers of methylionones; origin: Firmenich SA, Switzerland
⁴⁾1-(octahydro-2,3,8,8-tetramethyl-2-naphtalenyl)-1-ethanone; origin: IFF, USA
⁵⁾4-(1,1-dimethylethyl)-1-cyclohexyl acetate; origin: Firmenich SA, Switzerland
⁶⁾4/3-(4-hydroxy-4-methylpentyl)-3-cyclohexene-1-carbaldehyde; origin: IFF, USA
⁷⁾3-methyl-cyclopentadec-4/5-enone; origin: Firmenich SA, Switzerland
⁸⁾methyl dihydrojasmonate; origin: Firmenich SA, Switzerland
⁹⁾Methyl cedryl ketone; origin: IFF, USA

The addition of 100 parts by weight of (2E,4E)-2,4-hexadienyl salicylate to the above described composition strongly supported and developed the floral character of the scent thanks to its mimosa character. Furthermore, the addition of this compound introduced also a well harmonised vanilla, frangipane connotation. The result was a fairly natural and pleasant new scent.

On the other side, addition of the same amount of sorbol isobutyrate introduced a strong apple character which did not married the basic floral note of the original fragrance. Furthermore, the overall scent became more aggressive due to the presence of a strong galbanum note.

### Example 3

### Preparation of a Perfuming Composition

A perfuming composition of the fruity-green-pear and balsamic type was prepared by admixing the following ingredients:

| | Parts |
|---|---|
| Ingredient | by weight |
| Hexyl acetate | 45 |
| Ethyl acetoacetate | 45 |
| Bourgeonal ®¹⁾ | 5 |
| 10%* Damascenone | 10 |
| 10%* Damascone alpha | 15 |
| Dihydromyrcenol | 125 |
| 10%* Dynascone ®²⁾ | 15 |
| Ethyl (2E,4Z)-2,4-decadienoate | 45 |
| Ethyl linalol | 75 |
| 10%* Galbanum essential oil | 5 |
| Hedione ®³⁾ HC | 80 |
| Heliopropanal⁴⁾ | 75 |
| Iralia Total ®⁵⁾ | 30 |
| Lilial ®⁶⁾ | 50 |
| Linalol | 100 |
| Hedione ®⁷⁾ | 200 |
| 1%* Methyl octyne carbonate | 25 |
| 2,4-Dimethyl-3-cyclohexene-1-carbaldehyde | 5 |
| | 950 |

*in dipropyleneglycol
¹⁾3-(4-tert-butylphenyl)-propanal; origin: Quest, The Netherlands
²⁾1-(5,5-dimethyl-1-cyclohexen-1-yl)-4-penten-1-one; origin: Firmenich SA, Switzerland
³⁾high cis methyl dihydrojasmonate; origin: Firmenich SA, Switzerland
⁴⁾3-(1,3-benzodioxol-5-yl)-2-methylpropanal; origin: Firmenich SA, Switzerland
⁵⁾mixture of isomers of methylionones; origin: Firmenich SA, Switzerland
⁶⁾3-(4-tert-butylphenyl)-2-methylpropanal; origin: Givaudan-Roure SA, Vernier, Switzerland
⁷⁾methyl dihydrojasmonate; origin: Firmenich SA, Switzerland

The addition of 50 parts by weight of (E,E)-2,4-hexadienyl (Z)-3-hexenoate to the above-mentioned perfuming composition reinforced the fruity pear aspect of the fragrance and imparted also a natural tone. This addition transformed also the classical green aspect by imparting a sparkling and tonic nuance.

On the other side, addition of the same amount of sorbol isobutyrate introduced a pronounced disequilibrium by introducing a marked apple note, masking the pear character of the original composition. Furthermore, to the contrary of the invention's compound, the green note was also transformed by the introduction of an inelegant oily, resin and mastic note, typical of galbanum-like compound.

1-7. (canceled)

8. A method to confer, enhance, improve or modify the odor properties of a perfuming composition or of a perfumed article, which method comprises adding to said composition or article an effective amount of at least a compound of formula 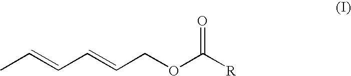
wherein R represents a linear C₅ alkenyl or alkadienyl group, a 6-hydroxy-phenyl group or a (C₆H₄)CH₂CH₂ group.

9. The method according to claim 8, wherein R represents a C₅ alkenyl group or a 6-hydroxy-phenyl group.

10. A compound of formula 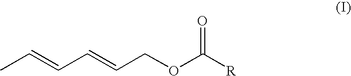
wherein R represents a linear C₅ alkenyl group, a 6-hydroxy-phenyl group or a (C₆H₄)CH₂CH₂ group.

11. A compound according to claim 10, specifically as (2E,4E)-2,4-hexadienyl salicylate, (E,E)-2,4-hexadienyl (Z)-3-hexenoate or (2E,4E)-2,4-hexadienyl 3-phenylpropanoate.

12. A perfuming composition comprising:
i) as perfuming ingredient, at least a compound of formula 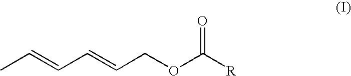
wherein R represents a linear C₅ alkenyl or alkadienyl group, a 6-hydroxy-phenyl group or a (C₆H₄)CH₂CH₂ group;
ii) at least one ingredient selected from the group consisting of a perfumery carrier and a perfumery base; and
iii) optionally at least one perfumery adjuvant.

13. A perfumed article comprising:
i) as perfuming ingredient, at least one compound of formula 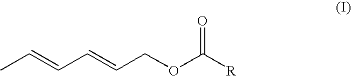
wherein R represents a linear C₅ alkenyl or alkadienyl group, a 6-hydroxy-phenyl group or a (C₆H₄)CH₂CH₂ group; and
ii) a consumer product base.

14. A perfumed article according to claim 13, wherein the consumer product base is a solid or liquid detergent, a fabric softener, a perfume, a cologne or after-shave lotion, a perfumed soap, a shower or bath salt, mousse, oil or gel, a hygiene product, a hair care product, a shampoo, a body-care product, a deodorant or antiperspirant, an air freshener, a cosmetic preparation, a fabric refresher, an ironing water, a paper, a wipe or a bleach.
